(19) United States
(12) Patent Application Publication
(10) Pub. No.: US 2009/0113607 A1
(43) Pub. Date: May 7, 2009

(54) Connection of goggle and mask

(75) Inventor: Sheng-Jen Lian, Taichung County (TW)

Correspondence Address: Sheng-Jen Lian, P.O.Box 44-2049, Taipei, 10668, TW

(73) Assignee: Yao-Gwo Gan

(21) Appl. No.: 11/979,379

(22) Filed: Nov. 2, 2007

(51) Int. Cl. A61F 9/02

(52) U.S. Cl. 2/427

(57) ABSTRACT

A mask includes a body having a through hole with which a goggle is engaged. Two first holes are defined in the body and located on two ends of the through hole. The goggle includes two side parts which are located on inside of the through holes. Two positioning plates each have a first positioning hole and a second positioning hole which communicates with the first positioning hole. Two locking members each have a protrusion which extends through the first hole, the side part and the first positioning hole, the positioning plates are then pulled to shift the protrusions to be engaged with the second positioning holes.

### BACKGROUND OF THE INVENTION

(1) Field of the Invention

The present invention relates to a mask with a removable goggle which is easily connected to or removed from the mask.

(2) Description of the Prior Art

A conventional mask for paint ball games generally includes a goggle which is fixed to the mask so as to protect wearers' eyes from being injured by the paint balls which moves at high speed and the paint splashes on the goggle during the game. The goggle cannot be separated from the mask so that after each game, the mask has to be cleaned up to remove the paint on the mask and goggle. However, the goggle gets wet after being cleaned so that it is not suitable to wear immediately. A lot of masks are needed to be replaced for different players in the following games. Besides, when the lenses are worn, the goggles have to be replaced in whole.

U.S. Pat. No. 6,948,813 discloses a goggle which is removably connected to the mask by using pivotable protrusion which is engaged with a groove in the mask. The protrusion is easily worn out after frequent engaging and disengaging with the groove. Once the protrusion is worn out, the goggle can be disengaged from the mask unexpectedly. Furthermore, the groove in the mask occupies a lot space and this weakens the structural strength of the mask.

The present invention intends to provide a connection of the goggle and the mask wherein two locking members are used to lock the mask and the goggle. The locking members each have a protrusion which is first inserted into a large hole and then shifted to be engaged with a small hole. The goggle can be easily connected to the mask and the connection involves less number of parts.

### SUMMARY OF THE INVENTION

The present invention relates to a mask which comprises a body having a through hole defined therethrough with which a goggle is engaged. Two first holes are defined in the body and located on two ends of the through hole. The goggle includes tow side parts and each have a second hole. Two positioning plates each have a first positioning hole and a second positioning hole which communicates with the first positioning hole. Two locking members each have a protrusion which extends through the first hole, the second hole in the side part and the first positioning hole, the positioning plates are then pulled to shift the protrusions to be engaged with the second positioning holes.

The present invention will become more obvious from the following description when taken in connection with the accompanying drawings which show, for purposes of illustration only, a preferred embodiment in accordance with the present invention.

### BRIEF DESCRIPTION OF THE DRAWINGS

FIG. 1 is an exploded view to show the mask with a goggle of the present invention;

FIG. 2 is a perspective view to show the mask with the goggle of the present invention;

FIG. 3 is a cross sectional view, taken along line A-A in FIG. 2;

FIG. 3A is an enlarged view to show the groove in the protrusion on the locking member and the shoulder of the second hole in the positioning plate, and

FIG. 4 shows the protrusion is shifted to be engaged with the second hole when the positioning plate is pulled.

### DETAILED DESCRIPTION OF THE PREFERRED EMBODIMENTS

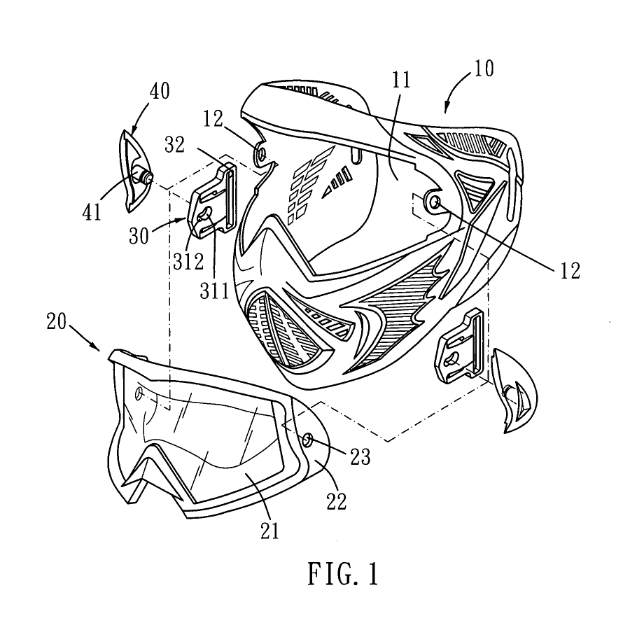

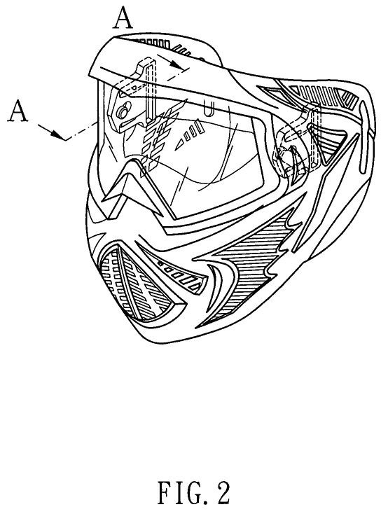

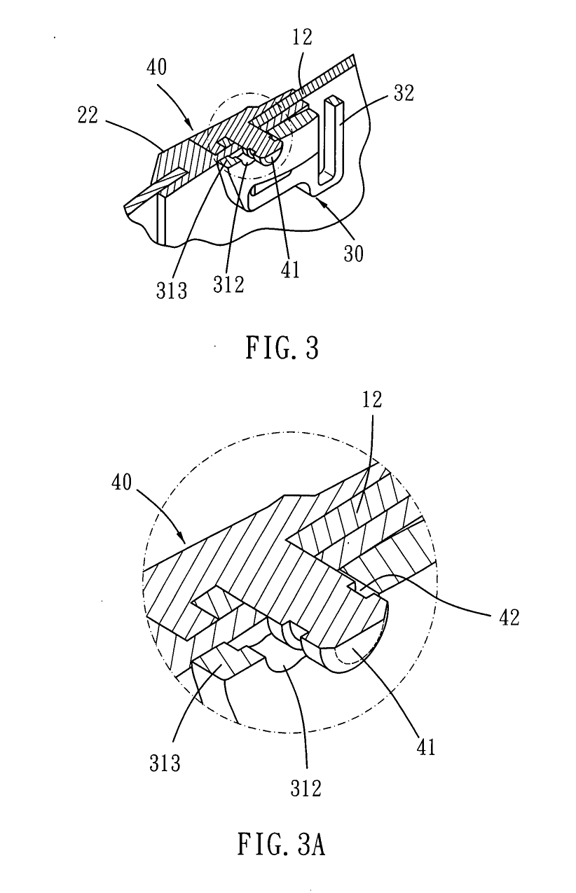

Referring to FIGS. 1, 2, 3 and 3A, the mask of the present invention comprises a body 10 which shaped to be suitable mounted to user's face and has a through hole 11 defined therethrough. Two lugs extend from two inner ends of the through hole 11 and each lug has a first hole 12.

A goggle 20 includes a lens 21 connected thereto and is engaged with the through hole 11. The goggle 20 includes two side parts 22 and each side part 22 has a second hole 23.

Two positioning plates 30 each have a first positioning hole 311 and a second positioning hole 312 which communicates with the first positioning hole 311. The inner diameter of the first positioning hole 311 is larger than the inner diameter of the second positioning hole 312. Each second positioning hole 312 includes a shoulder 313. Each positioning plate 30 further has a pull end 32 to which a belt is connected, and two slots 23 are defined through each positioning plate 30, the first and second positioning holes 311, 312 are located between the two slots 23. One of the slots is connected with a strap.

Two locking members 40 are located on outside of the body 10 and each have a protrusion 41 which has a groove 42 defined radially in an outer periphery thereof and the shoulder 313 of the second positioning hole 312 is sized to be engaged with the groove 42.

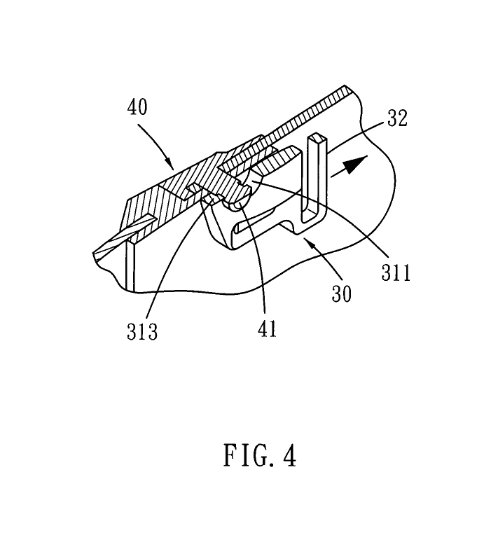

As shown in FIG. 4, when mounting the goggle 20 to the body 10 of the mask, the two side parts 22 are located at insides of the two lugs and the two protrusions 41 extend through the first holes 12, the second holes 23 in the side parts 22 and the first positioning hole 311. The positioning plates 30 are then pulled to shift the protrusions 41 to be engaged with the second positioning holes 312 by engaging the shoulders 313 with the grooves 42 of the protrusions 41.

The sequence of the side parts 22, the lugs of the body 10 can also be exchanged to put the side parts 22 located on outside of the first holes 12.

The goggle 20 is conveniently connected to the body 10 of the mask and the users simply pull the belt connected between the two positioning plates 30 to quickly secure the goggle 20 on the body 10 of the mask.

While we have shown and described the embodiment in accordance with the present invention, it should be clear to those skilled in the art that further embodiments may be made without departing from the scope of the present invention.

What is claimed is:

1. A mask comprising:
a body having a through hole defined therethrough and two first holes defined in the body and located on two ends of the through hole; a goggle engaged with the through hole and having two side parts; two positioning plates each having a first positioning hole and a second positioning hole which communicates with the first positioning hole, an inner diameter of the first positioning hole being larger than an inner diameter of the second positioning hole, and two locking members each having a protrusion which extends through the first hole, the side part and the first positioning hole, the positioning plates being pulled to shift the protrusions to be engaged with the second positioning holes.

2. The mask as claimed in claim 1, wherein the goggle includes a lens connected thereto.

3. The mask as claimed in claim 1, wherein each of the protrusions includes a groove defined radially in an outer periphery thereof, each second positioning hole includes a shoulder which is engaged with the groove.

4. The mask as claimed in claim 1, wherein each positioning plate includes a pull end to which a belt is connected.

5. The mask as claimed in claim 1, wherein each positioning plate includes two slots defined therethrough and the first and second positioning holes are located between the two slots, one of the slots is connected with a strap.

6. The mask as claimed in claim 1, wherein the side parts each have a second hole and are located on inside of the first holes.

7. The mask as claimed in claim 1, wherein the side parts each have a second hole and are located on outside of the first holes.
